(19) United States
(12) Patent Application Publication
(10) Pub. No.: US 2004/0242589 A1
(43) Pub. Date: Dec. 2, 2004

(54) 3-arylsulfonyl-7-piperzinyl-indoles-benzofurans and -benzothiophenes with 5-ht6 receptor affinity for treating cns disorders

(76) Inventors: Steven Mark Bromidge, Verona (IT); Christopher Norbert Johnson, Essex (GB); Gregor James MacDonald, Essex (GB); Mervyn Thompson, Essex (GB); David R Witty, Essex (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/486,068

(22) PCT Filed: Aug. 5, 2002
(86) PCT No.: PCT/EP02/08719

(30) Foreign Application Priority Data
Aug. 7, 2001 (GB) 0119242.6
Feb. 12, 2002 (GB) 0203300.9

(51) Int. Cl. A61K 31/496; C 07D 4 3/14; C 07D 4 5/14; C 07D 4 9/14

(52) U.S. Cl. 514/252.13; 514/253.08; 514/253.1; 514/254.09; 514/254.11; 544/360; 544/373; 544/359

(57) ABSTRACT

The invention relates to novel compounds of formula (I) having affinity for the 5-HT₆ receptor preparation, to compositions containing them and their use in the treatment of various disorders, including CNS disoders.

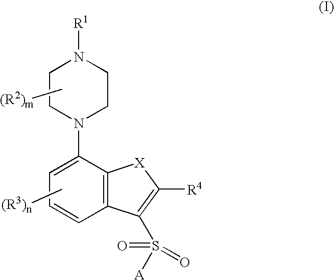

This invention relates to novel indole compounds having pharmacological activity, processes for their preparation, to compositions containing them and to their use in the treatment of CNS and other disorders.

WO 98/27081 discloses a series of aryl sulphonamide compounds that are said to be 5-HT₆ receptor antagonists and which are claimed to be useful in the treatment of various CNS disorders. GB-2341549, WO 99/47516 and WO 99/65906 all disclose a series of indole derivatives that are claimed to 5-HT₆ receptor affinity.

A structurally novel class of compounds has now been found which also possess 5-HT₆ receptor affinity. The present invention therefore provides, in a first aspect, a compound of formula (1) or a pharmaceutically acceptable salt thereof:

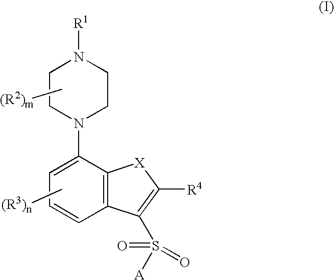

wherein:

R¹ and R² independently represent hydrogen or C₁₋₆ alkyl or R¹ is linked to R² to form a group (CH₂)₂, (CH₂)₃ or (CH₂)₄;

R³ independently represents hydrogen, halogen, cyano, —CF₃, —CF₃O, C₁₋₆ alkyl, C₁₋₆ alkoxy, C₁₋₆ alkanoyl or a group —CONR⁵R⁶;

R⁴ represents hydrogen or C₁₋₆ alkyl;

R⁵ and R⁶ independently represent hydrogen or C₁₋₆ alkyl or together may be fused to form a 5- to 7-membered aromatic or non-aromatic heterocyclic ring optionally interrupted by an O or S atom;

m represents an integer from 1 to 4, such that wherein m is an integer greater than 1, said R² groups may optionally be linked to form a group CH₂, (CH₂)₂ or (CH₂)₃;

n represents an integer from 1 to 3;

X represents NH, N-C₁₋₆ alkyl, O or S;

A represents a group —Ar¹ or —Ar²Ar³;

Ar¹, Ar² and Ar³ independently represent an aryl group or a heteroaryl group, both of which may be optionally substituted by one or more substituents which may be the same or different, and which are selected from the group consisting of halogen, hydroxy, cyano, nitro, trifluoromethyl, trifluoromethoxy, C₁₋₆ alkyl, trifiuoromethanesulfonyloxy, pentafluoroethyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, arylsulfonyl, arylsulfonyloxy, arylsulfonylC₁₋₆ alkyl, C₁₋₆ alkylsulfonamido, C₁₋₆ alkylamido, C₁₋₆ alkylsulfonamidoC₁₋₄ alkyl, C₁₋₆, alkylamidoC₁₋₆ alkyl, arylsulfonamido, arylcarboxamido, arylsulfonamidoC₁₋₆ alkyl, arylcarboxamidoC₁₋₆ alkyl, aroyl, aroylC₁₋₆ alkyl, arylC₁₋₆ alkanoyl, or a group CONR⁷R⁸ or SO₂NR⁷R⁸, wherein R⁷ and R⁸ independently represent hydrogen or C₁₋₆ alkyl or together may be fused to form a 5- to 7-membered aromatic or non-aromatic heterocyclic ring optionally interrupted by an O or S atom;

or solvates thereof.

Alkyl groups, whether alone or as part of another group, may be straight chain or branched and the groups alkoxy and alkanoyl shall be interpreted similarly. Alkyl moieties are more preferably C₁₋₄ alkyl, eg. methyl or ethyl. The term ‘halogen’ is used herein to describe, unless otherwise stated, a group selected from fluorine, chlorine, bromine or iodine.

The term “aryl” includes phenyl and naphthyl.

The term “heteroaryl” is intended to mean a 5-7 membered monocyclic aromatic or a fused 8-11 membered bicyclic aromatic ring containing 1 to 3 heteroatoms selected from oxygen, nitrogen and sulphur. Suitable examples of such monocyclic aromatic rings include thienyl, furyl, pyrrolyl, triazolyl, imidazolyl, oxazolyl, thiazolyl, oxadiazolyl, isothiazolyl, isoxazolyl, thiadiazolyl, pyrazolyl, pyrimidyl, pyridazinyl, pyrazinyl and pyridyl. Suitable examples of such fused aromatic rings include benzofused aromatic rings such as quinolinyl, isoquinolinyl, quinazolinyl, quinoxalinyl, cinnolinyl, naphthyridinyl, indolyl, indazolyl, pyrrolopyridinyl, benzofuranyl, benzothienyl, benzimidazolyl, benzoxazolyl, benzisoxazolyl, benzothiazolyl, benzisothiazolyl, benzoxadiazolyl, benzothiadiazolyl and the like. Heteroaryl groups, as described above, may be linked to the remainder of the molecule via a carbon atom or, when present, a suitable nitrogen atom.

It will be appreciated that wherein the above mentioned aryl or heteroaryl groups have more than one substituent, said substituents may be linked to form a ring, for example a carboxyl and arine group may be linked to form an amide group.

When Ar¹, Ar² or Ar³ are substituted they are preferably substituted by 1 or 2 substituents.

Preferably, R¹ represents hydrogen or methyl. Most preferably, R₁ represents hydrogen.

Preferably, R² represents hydrogen.

Preferably, R³ represents hydrogen or halogen (such as chlorine, eg. 4-, 5- or 6-chlorine). Most preferably, R³ represents hydrogen.

Preferably, R⁴ represents hydrogen or methyl. Most preferably, R⁴ represents hydrogen.

Preferably, X represents NH, N—CH₃, O or S. Most preferably, X represents N—CH₃.

Preferably, m represents 1.

Preferably n represents 1 or 2. Most preferably, n represents 1.

Preferably, A represents Ar¹.

When Ar¹ represents a heteroaryl group it is preferably N-linked indole, such as 1H-indol-1-yl, or C-linked pyridyl such as 2-pyridyl, each of which may be optionally substituted.

When Ar¹ represents an aryl group it is preferably optionally substituted phenyl.

Preferably, Ar¹ is optionally substituted with one or more halogen (particularly 2- or 3-chloro and 2-, 3- and 4-fluoro), cyano (particularly 2-cyano), trifluoromethyl (particularly 2-trifluoromethyl), methyl, trifluoromethoxy or acetyl groups. More preferably, Ar¹ is phenyl optionally substituted with one or more halogen particularly 2 or 3-chloro and 2, 3 and 4-fluoro), cyano (particularly 2-cyano) or trifluoromethyl (particularly 2-trifluoromethyl) groups.

Most preferably, Ar¹ represents phenyl substituted by one halogen, such as chlorine (especially 3-chlorine).

Preferred compounds according to the invention include examples E1-E26 as shown below, or a pharmaceutically acceptable salt thereof.

The compounds of formula (1) can form acid addition salts thereof. It will be appreciated that for use in medicine the salts of the compounds of formula (1) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. The present invention includes within its scope all possible stoichiometric and non-stoichiometric forms.

The compounds of formula (I) may be prepared in crystalline or non-crystalline form, and, if crystalline, may optionally be solvated, eg. as the hydrate. This invention includes within its scope stoichiometric hydrates as well as compounds containing variable amounts of water.

Certain compounds of formula (I) are capable of existing in stereoisomeric forms (e.g. diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

The present invention also provides a process for the preparation of a compound of formula (I) or a pharmaceutically acceptable salt thereof, which process comprises:

(a) reacting a compound of formula (II)

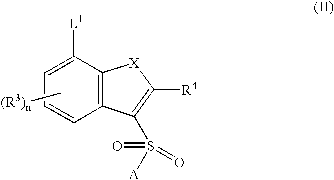

with a compound of formula (E)

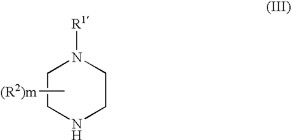

wherein R^{1′} is as defined above for R¹ or an N-protecting group, R², R³, R⁴, A, X, m and n are as defined above and L¹ represents a suitable leaving group, such as a halogen atom (eg. bromine) or a trifluoromethylsulfonyloxy group; or

(b) forming a compound of formula (I) wherein R² represents hydrogen which comprises reacting a compound of formula (IV)

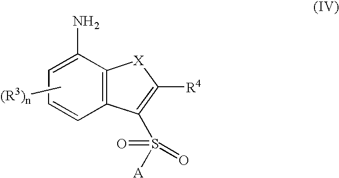

with a compound of formula (V)

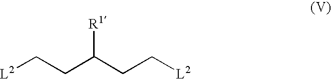

wherein R^{1′} is as defined above for R¹ or an N-protecting group, R³, R⁴, X, A and n are as defined above and L² represents a suitable leaving group, such as a halogen atom; or

(c) oxidation of a compound of formula (VI)

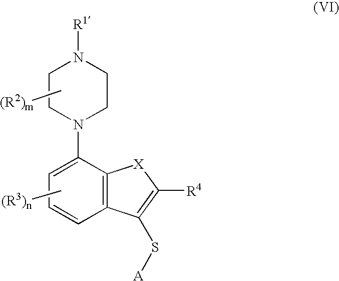

wherein R^{1′} is as defined above for R¹ or an N-protecting group and R², R³, R⁴, A, m, n and X are as defined above; or

(d) deprotecting a compound of formula (I) which is protected;

and optionally thereafter interconversion to other compounds of formula (1).

The N-protecting group used may be any conventional group eg. t-butyloxycarbonyl (Boc) or benzyloxycarbonyl.

Process (a) typically comprises the use of a suitable base, such as sodium t-butoxide or cesium carbonate in the presence of a palladium catalyst (eg. palladium (1H) acetate or tris(dibenzylideneacetone)dipalladium (0) in the presence of a suitable ligand such as 2,2′-bis(diphenylphosphino)-1,1′-binaphthyl (BINAP) in a suitable solvent such as dioxan or dimethylformamide.

Process (b) typically comprises the use of a suitable base, such as sodium carbonate and the use of a suitable solvent such as n-butanol.

Process (c) typically comprises the use of an oxidising agent, eg. hydrogen peroxide or a peracid reagent, such as peracetic or 3-chloroperbenzoic acid.

In process (d), examples of protecting groups and the means for their removal can be found in T. W. Greene ‘Protective Groups in Organic Synthesis’ (J. Wiley and Sons, 1991). Suitable amine protecting groups include sulphonyl (e.g. tosyl), acyl (e.g. benzyloxycarbonyl or t-butoxycarbonyl) and arylalkyl (e.g. benzyl), which may be removed by hydrolysis or hydrogenolysis as appropriate. Other suitable amine protecting groups include trifluoroacetyl (—COCF₃) which may be removed by base catalysed hydrolysis or a solid phase resin bound benzyl group, such as a Merrifield resin bound 2,6-dimethoxybenzyl group (Ellman linker), which may be removed by acid catalysed hydrolysis, for example with trifluoroacetic acid. A further amine protecting group includes methyl which may be removed using standard methods for N-dealkylation (eg. 1-chloroethyl chloroformate under basic conditions followed by treatment with methanol).

Interconversion of compounds of formula (I) may be performed using conventional interconversion procedures such as epimerisation, oxidation, reduction, alkylation, nucleophilic aromatic substitution, ester hydrolysis or amide bond formation. For example, N-dealkylation of a compound of formula (I) wherein R¹ represents an alkyl group to give a compound of formula (I) wherein R¹ represents hydrogen. A further example of interconversion may include the alkylation of a compound of formula (I) wherein X represents NH to a compound of formula (I) wherein X represents N—C₁₋₆ alkyl. It will be appreciated that such interconversion may be interconversion of protected derivatives of formula (I) which may subsequently be deprotected following interconversion.

Compounds of formula (IV) may be prepared according to the following process:

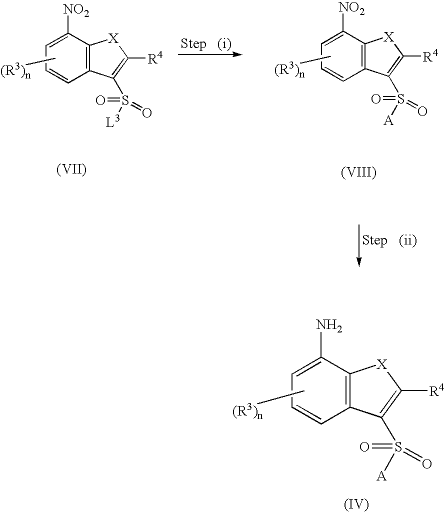

wherein R³, R⁴, X, A and n are as defined above and L³ represents a suitable leaving group, such as a halogen atom (eg. a fluorine or chlorine atom).

Step (i) typically comprises the reaction of a compound of formula (VII) with a compound of formula A-M, wherein A is as defined above and M represents a metal containing moiety, such as sodium, lithium, magnesium halide or zinc halide.

Step (ii) typically comprises reduction of a compound of formula (VIM), for example using hydrogenation in the presence of a suitable catalyst such as palladium on carbon.

It will be appreciated that compounds of formula (II) may be prepared in an analogous process to that of step (i) above.

Compounds of formula (IV) where X represents 0 may be more advantageously prepared according to the following process:

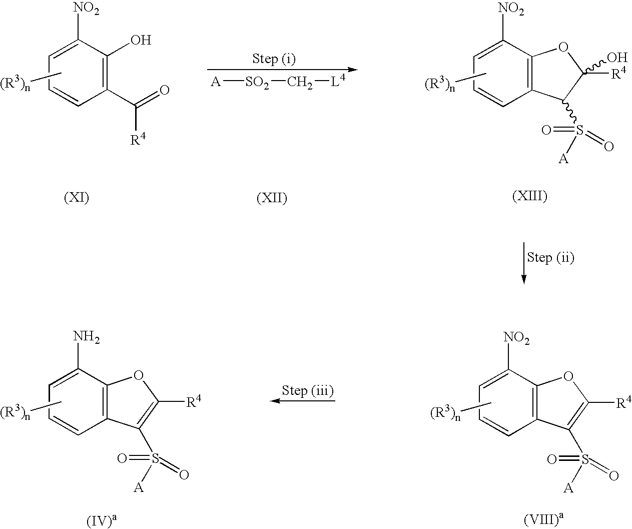

wherein R³, R⁴, A and n are as defined above and L⁴ represents a suitable leaving group, such as a halogen (eg. chlorine).

Step (i) comprises reaction of a compound of formula (XI) with a compound of formula (XII) in the presence of a base such as potassium t-butoxide in a suitable solvent such as N,N-dimethylformamide at an appropriate temperature, e.g. −40° C.

Step (ii) comprises use of a strong acid such as sulfuric acid in a suitable solvent such as acetic acid at an appropriate temperature, e.g. 60° C.

Step (iii) typically comprises reduction of a compound of formula (VIII)^{a}, for example using hydrogenation in the presence of a suitable catalyst such as palladium on carbon.

Other compounds of formula (VIII) are known or may be prepared by methods analogous to those described in the literature or analogous to those described above.

Compounds of formula (VI) where X is NH may be prepared according to the following process:

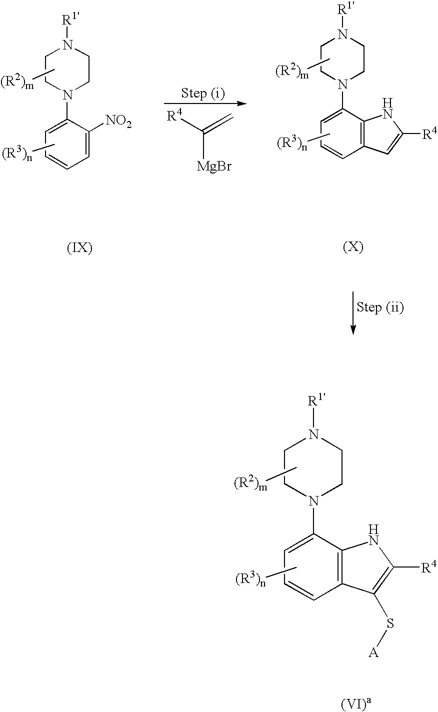

wherein R^{1′} is as defined above for R¹ or an N-protecting group, eg. benzyloxycarbonyl (Boc), and R², R³, R⁴, A, m and n are as defined above.

Step (i) typically comprises the use of a solvent eg. tetrahydrofuran at a suitable temperature, eg. −40° C.

Step (ii) typically comprises the use of a base, eg. sodium hydride followed by reaction of a compound of formula A-S—S-A, wherein A is as defined above in a solvent such as N,N-dimethylformamide at a suitable temperature, eg. 20° C.

Compounds of formula (VI) wherein X represents N—C₁₋₆ alkyl may be prepared according to the following process:

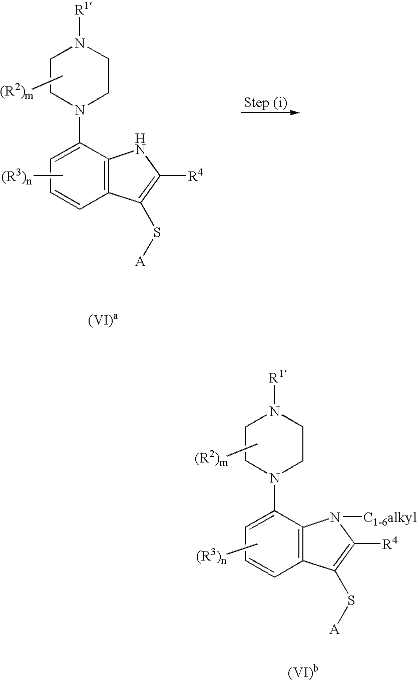

Step (i) typically comprises the use of a base, eg. sodium hydride, potassium hydroxide or sodium hydroxide followed by reaction with an appropriate C₁₋₆alkylating agent such as methyl iodide or dimethyl sulphate in a solvent such as N,N-dimethylformamide or acetone at a suitable temperature e.g. 20° C.

Compounds of formula (II) wherein X represents O or S may be prepared according to the following process:

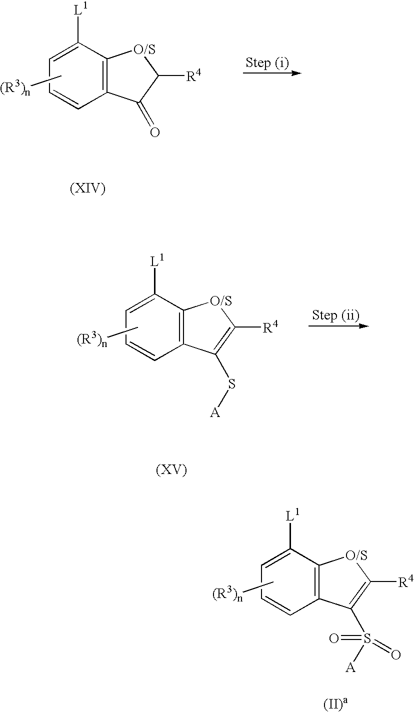

wherein R³, R⁴, A, n and L¹ are as defined above.

Step (i) typically comprises reaction with a compound A-SH, where A is as defined above, in a suitable solvent such as benzene in the presence of an acid such as p-toluenesulfonic acid at an appropriate temperature (e.g. at reflux).

Step (ii) typically comprises the use of an oxidising agent such as monoperoxyphthalic acid or 3-chloroperbenzoic acid in a suitable solvent system, e.g. a mixture of methanol and dichloromethane.

Other compounds of formula (II) are known or may be prepared by methods analogous to those described in the literature or analogous to those described above.

Compounds of formula (III), (V), (VII), (IX), (XI), (XII) and (XIV) are either known or may be prepared in accordance with known or analogous procedures. It will be appreciated that compounds of formula (XIV) may also exist as the hydroxy tautomer.

Pharmaceutically acceptable salts may be prepared conventionally by reaction with the appropriate acid or acid derivative.

Compounds of formula (I) and their pharmaceutically acceptable salts have 5-HT₆ receptor activity and are believed to be of potential use in the treatment of certain CNS disorders such as anxiety, depression, epilepsy, obsessive compulsive disorders, migraine, cognitive memory disorders (e.g. Alzheimers disease, age related cognitive decline and mild cognitive impairment), Parkinsons Disease, ADHD (Attention Deficit Disorder/Hyperactivity Syndrome), sleep disorders (including disturbances of Circadian rhythm), feeding disorders such as anorexia and bulimia, panic attacks, withdrawal from drug abuse such as cocaine, ethanol, nicotine and benzodiazepines, schizophrenia, and also disorders associated with spinal trauma and/or head injury such as hydrocephalus. Compounds of the invention are also expected to be of use in the treatment of certain GI (gastrointestinal) disorders such as EBS (Irritable Bowel Syndrome). Compounds of the invention are also expected to be of use in the treatment of obesity.

Thus the invention also provides a compound of formula (I) or a pharmaceutically acceptable salt thereof, for use as a therapeutic substance, in particular in the treatment or prophylaxis of the above disorders. In particular the invention provides for a compound of formula (I) or a pharmaceutically acceptable salt thereof, for use in the treatment of depression, anxiety, Alzheimers disease, age related cognitive decline, ADHD, obesity, mild cognitive impairment and schizophrenia.

The invention further provides a method of treatment or prophylaxis of the above disorders, in mammals including humans, which comprises administering to the sufferer a therapeutically effective amount of a compound of formula (I) or a pharmaceutically acceptable salt thereof.

In another aspect, the invention provides the use of a compound of formula (I) or a pharmaceutically acceptable salt thereof in the manufacture of a medicament for use in the treatment or prophylaxis of the above disorders.

In order to use the compounds of formula (I) in therapy, they will normally be formulated into a pharmaceutical composition in accordance with standard pharmaceutical practice. The present invention also provides a pharmaceutical composition, which comprises a compound of formula (I) or a pharmaceutically acceptable salt thereof, and a pharmaceutically acceptable carrier.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusable solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colourants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10 to 60% by weight, of the active material, depending on the method of administration.

The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 0.05 to 20.0 mg, for example 0.2 to 5 mg; and such unit doses may be administered more than once a day, for example two or three times a day, so that the total daily dosage is in the range of about 0.5 to 100 mg; and such therapy may extend for a number of weeks or months.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the invention.

Description 1: 3-(1H-Indole-1-sulfonyl)-7-nitro-1H-indole (D1)

To a stirred suspension of sodium hydride (0.46 g, 11.5 mmol, 60% suspension in oil) in dimethylformamide (10 ml) was added dropwise 1H-indole (1.34 g, 11.5 mmol) as a solution in dimethylformamide (5 ml). After stirring for 20 minutes, a solution of 7-nitro-1H-indole-3-sulfonyl chloride (Mandur et al. Chem. Heterocycl. Compd. (Engl. Transl.); 1990, 26, 1116-1120, 2.0 g, 7.7 mmol) in dimethylformamide (5 ml) was added dropwise and the resulting mixture was left to stir for 15 hours. The reaction mixture was then diluted with dichloromethane (100 ml) and washed with water (2×50 ml), brine (50 ml), dried (MgSO₄) and filtered. The filtrate was concentrated in vacuo and purified by column chromatography over silica gel, eluting with dichloromethane to afford the title compound (D1) (88 mg, 77%), MS: m/z (M−H)⁻ 339, C₁₆H₁₁N₃O₄S requires 340.

Description 2: 3-(1H-Indole-1-sulfonyl)-1H-7-indol-7-ylamine (D2)

3-(1H-Indole-1-sulfonyl)-7-nitro-1H-indole (D1) (1.0 g, 2.9 mmol) was dissolved in ethanol (50 ml) followed by addition of palladium catalyst (0.2 g, 10% Pd/C). The resulting mixture was stirred under an atmosphere of hydrogen for 15 hours followed by filtration (CELITE) and in vacuo removal of solvent. This provided the title compound (D2) (0.73 g, 80%), MS: m/z (M−H)⁻ 309, C₁₆H₁₃N₃O₂S requires 310.

Description 3: 4-(1H-Indol-7-yl)piperazine-1-carboxylic acid tert-butyl ester (D3)

A solution of 4-(2-nitrophenyl)piperazine-1-carboxylic acid tert-butyl ester [(For synthesis see: Tetrahedron Lett. 1997, 38 (23), 4091-4094), (33.1 g, 0.108 mol)] in THF (750 ml) under argon was cooled to −45° C. To this cooled solution was added via dropping funnel a 1M solution of vinyl magnesium bromide in THF (345 ml, 0.345 mol) over 20 minutes. The mixture was stirred at this temperature for 40 minutes followed by slow addition of sat. NH₄Cl solution and the resulting mixture extracted with dichloromethane. The organic phase was washed with water, dried (MgSO₄), filtered and the solvent evaporated. The crude material was purified by column chromatography on silica gel (acetone/toluene gradient) to afford the title compound (D3) (3.55 g, 11%), δ_{H} (CDCl₃) 1.50 (9H, s), 3.06 (4H, t, J=5.0 Hz), 3.65 (4H, t, J=4.8 Hz), 6.56 (1H, m), 6.83 (1H, d, J=7.5 Hz), 7.07 (1H, t, J=7.7 Hz), 7.20 (1H, m), 7.39 (1H, d, J=7.9 Hz), 8.28 (1H, br s).

Description 4: 4-[3-(3-Chlorophenyl)sulfonyl-1H-indol-7-yl]piperazine-1-carboxylic acid tert-butyl ester (D4)

To a suspension of sodium hydride (0.20 g, 5.0 mmol), in DMF (15 ml) under argon was added 4-(1H-indol-7-yl)piperazine-1-carboxylic acid tert-butyl ester (D3) (1.0 g, 3.3 mmol) in portions over 5 minutes. After complete addition, the mixture was stirred at room temperature for a further 10 minutes and a solution of bis-(3-chlorophenyl) disulfide (1.04 g, 3.6 mmol) in DMF (5 ml) was then added over 5 minutes. The solution was stirred for 2 hours followed by careful addition of water (100 ml) and then diethyl ether (100 ml). The organic layer was separated, washed with water (100 ml), dried (MgSO₄) and concentrated in vacuo to provide 4-[3-(3-chlorophenyl)sulfanyl-1-methyl-1H-indol-7-yl]piperazine-1-carboxylic acid tert-butyl ester (1.16 g, 79%). This was dissolved in dichloromethane (40 ml) and to it added 3-chloroperbenzoic acid (2.1 g, 9.1 mmol) portion wise over 5 minutes. The resulting mixture was stirred at ambient temperature for 18 hours and then diluted with dichloromethane (40 ml), washed with saturated aqueous sodium metabisulfite (100 ml), sat. NaHCO₃ solution (100 ml), dried (MgSO₄) and concentrated in vacuo. The crude material was purified by column chromatography on silica gel, eluting with dichloromethane to afford the title compound (D4) (930 mg, 75%), δ_{H} (CDCl₃) 1.49 (9H, s), 3.00 (4H, t, J=5.1 Hz), 3.63 (4H, t, J=4.9 Hz), 6.94 (1H, d, J=7.0 Hz), 7.22 (1H, t, J=7.9 Hz), 7.40-7.47 (2H, m), 7.64 (1H, d, J=8.0 Hz), 7.89-7.93 (2H, m), 7.98 (1H, t, J=1.5 Hz), 9.13 (1H, br s).

Description 5: 4-[3-(3-Chlorophenyl)sulfonyl-1-methyl-1H-indol-7-yl]piperazine-1-carboxylic acid tert-butyl ester (D5)

A mixture of 4-[3-(3-chlorophenyl)sulfonyl-1H-indol-7-yl]piperazine-1-carboxylic acid tert-butyl ester (D4) (115 mg, 0.24 mmol) and KOH (17 mg, 0.3 mmol) in ethanol (2 ml) was stirred at room temperature for 30 minutes and the solvent removed in vacuo. The residue was dissolved in acetone (2 ml) and dimethylsulfate (23 μL, 0.24 mmol) added to the solution. The mixture was stirred at ambient temperature for 1 hour followed by addition of dichloromethane (20 ml). The organic phase was washed with water (10 ml), dried (MgSO₄) and concentrated in vacuo. Purification of the crude material by column chromatography on silica gel (petroleum ether (40-60)/ethylacetate gradient) gave the title compound (D5) (70 mg, 60%), δ_{H} (CDCl₃) 1.49 (9H, s), 2.85-2.91 (2H, m), 3.02-3.09 (4H, m), 4.12 (2H, m), 4.21 (3H, s), 7.05 (1H, d, J=6.7 Hz), 7.23 (1H, t, J=Hz), 7.36-7.46 (2H, m), 7.69 (2H, m), 7.91 (1H, m), 7.96 (1H, t, J=1.8 Hz).

Description 6: 1-(5-Chloro-2-nitro)phenyl-4-methylpiperazine (D6)

A solution of 4-chloro-2-fluoronitrobenzene [Fluorochem] (863 mg, 5 mmol), in dichloromethane (5 ml) was treated with triethylamine (505 mg, 5 mmol) and N-methylpiperazine (5 mmol, 500 mg) at 0° C. The solution was allowed to warm to room temperature, poured into water (500 ml), and extracted with diethyl ether (3×100 ml). The combined ether extracts were dried (MgSO₄), filtered and evaporated and the residue purified by flash chromatography (methanol—dichloromethane) on silica gel to give the title compound (D6) as a yellow solid (440 mg, 34%) δ_{H} (CDCl₃) 2.53 (3H, s), 2.57 (4H, t, J=4.8 Hz), 3.10 (4H, t, J=4.8 Hz), 6.96 (1H, dd, J=2.1 Hz, 8.7 Hz), 7.07 (1H, d, J=2.1 Hz), 7.76 (1H, d, J=8.7 Hz).

Description 7: 2,3-Dihydro-7-nitro-3-phenylsulfonyl-benzo[b]furan-2-ol (D7)

A solution of 2-hydroxy-3-nitrobenzaldehyde (2.0 g, 12 mmol) and chloromethylphenyl sulfone (4.6 g, 24 mmol) in N,N-dimethylformamide (160 ml) was added over 5 minutes to a stirred solution of potassium-t-butoxide (45.6 g, 400 mmol) in N,N-dimethylformamide (200 ml) at −40° C. After 2 h, the solution was warmed to room temperature and shaken with ethyl acetate (500 ml) and 0.5M hydrochloric acid (500 ml). The layers were separated and the organic phase dried (MgSO₄) and concentrated in vacuo to an oil. The oil was purified by chromatography over silica gel using a solvent gradient of acetone/toluene to afford the title compound (D7) as a gum (2.1 g, 55%). Mass Spectrum: m/z [M−H]⁻ 320 (C₁₄H₁₁NO₆S).

Description 8: 7-Nitro-3-phenylsulfonyl-benzo[b]furan (D8)

A solution of 2,3-dihydro-7-nitro-3-phenylsulfonyl-benzo[b]furan-2-ol (D7) (2.1 g, 6.5 mmol) in conc. sulfuric acid (specific gravity, 1.8) (20 ml) and glacial acetic acid (20 ml) was heated at 60° C. for 10 h. After cooling the reaction mixture in an ice bath, it was taken to pH 8 using 10% sodium hydroxide solution, and extracted with chloroform (2×150 ml). The combined organic extracts were dried (MgSO₄) and concentrated iii vacuo to afford the title compound (D8) as an oil (1.65 g, 83%).

δ_{H} (CDCl₃) 7.51-7.67 (4H, m), 8.06 (2H, d, J=7.4 Hz), 8.20 (1H, d, J=7.9 Hz), 8.26 (1H, d, J=8.1 Hz), 8.44 (1H, s).

Description 9: 3-Phenylsulfonyl-benzo[b]furan-7-ylamine (D9)

A solution of 7-nitro-3-phenylsulfonyl-benzo[b]furan (D8) (1.5 g, 5.0 mmol) in ethanol (110 ml) and N,N-dimethylformamide (40 ml) was stirred at ambient temperature with 10% palladium on carbon (1.5 g) under one atmosphere of hydrogen for 3 h. The mixture was filtered to remove the catalyst and the filtrate concentrated in vacuo to an oil. The oil was purified by chromatography over silica gel eluting with a solvent gradient of acetone/toluene to afford the title compound (D9) (0.51 g, 37%). Mass Spectrum: m/z [MH]⁺ 274 (C₁₄H₁₁NO₃S).

Description 10: 7-Bromo-3-phenylsulfanyl-benzo[b]thiophene (1)10)

A solution of 7-bromo-3-hydroxy-benzo[b]thiophene [J. Indian Chem. Soc. 43 (9) 597 1966] (1.15 g, 5.0 mmol), thiophenol (0.56 g, 5.0 mmol) and p-toluene sulfonic acid (0.1 g catalytic), in dry benzene (30 ml) was refluxed with the condensate passing through 3A molecular sieves (10 ml), in a Soxhlet device for 16 h. The solvent was removed and the residue was chromatographed on silica using pentane as eluant to afford the title compound (D10) (710 mg, 50% yield). δ_{H} (CDCl₃) 7.23-7.12 (5H, m), 7.25 (1H, t, J=8.1 Hz), 7.54 (1H, d, J=7.6 Hz), 7.75 (2H, m).

Description 11: 7-Bromo-3-phenylsulfonyl-benzo[b]thiophene (D11)

7-Bromo-3-phenylsulfanyl-benzo[b]thiophene (D10) (125 mg, 0.39 mmol) and magnesium mono-peroxyphthalate (268 mg, 0.43 mmol) were stirred at room temperature in methanol (5 ml) and dichloromethane (20 ml) for 16 h. The solvent was removed and the residue was triturated under dichloromethane then filtered. The solvent was removed to afford crude product which was purified by chromatography on silica using dichloromethane as eluant to afford the title compound (D11) (125 mg, 91% yield). 8H (CDCl₃) 7.36 (1H, t, J=7.9 Hz), 7.59-7.49 (4H, m), 8.08 (2H, dd, J=0.3 Hz, 8.4 Hz), 8.18 (1H, d, J=7.7 Hz), 8.50 (1H, s).

Description 12: 7-(4-tert-Butyloxycarbonyl-1-piperazinyl)-3-phenylsulfonyl-benzo[b]thiophene (1)12)

A catalyst suspension was prepared by sonicating palladium acetate (5 mg, 0.022 mmol), cesium carbonate (98 mg, 0.3 mmol), and BINAP [2,2′-bis(diphenylphosphino)-1,1′-binaphthyl] (20 mg, 0.032 mmol) in dioxane (5 ml) under argon for 45 mins at 35° C. 7-Bromo-3-phenylsulfonyl-benzo[b]thiophene, (D11) (66 mg, 0.19 mmol), and 1-tert-butyloxycarbonyl-piperazine (56 mg, 0.3 mmol) was added to the above catalyst and the stirred mixture heated to 100° C. under argon for 16 h. The mixture was filtered and the solvent removed. The crude product was purified by chromatography on silica with a pentane/ethyl acetate mixture (3:1), as eluant to afford the title compound (D12) (48 mg, 53% yield). δ_{H} (CDCl₃) 1.48 (9H, s), 3.08 (4H, m), 3.63 (4H, m), 7.00 (1H, d, J=7.6 Hz), 7.41 (1H, t, J=8.0 Hz), 7.51 (3H, m), 7.90 (1H, d, 3=8.0 Hz), 8.03 (2H, d, J=7.3 Hz), 8.44 (1H, s).

Descriptions 13 and 14: 7-(4-tert-Butyloxycarbonyl-1-piperazinyl)-6-chloro-3-phenylsulfonyl-benzo[b]thiophene (D13) and 7-(4-tert-butyloxycarbonyl-1-piperazinyl)₄ chloro-3-phenylsulfonyl-benzo[b]thiophene (D14)

To a solution of 7-(4-tert-butyloxycarbonyl-1-piperazinyl)-3-phenylsulfonyl-benzo[b]thiophene (D12) (244 mg, 0.53 mmol) in glacial acetic acid (10 ml) was added N-chlorosuccinimide (71 mg, 0.53 mmol) portionwise over 10 minutes. The reaction mixture was heated at 60° C. for 18 h under argon. The reaction mixture was then cooled to room temperature, diluted with dichloromethane (20 ml), water (10 ml) and adjusted to pH 8 with saturated sodium hydrogen carbonate solution. The organic layer was separated, washed with brine, dried (MgSO₄) and concentrated in vacuo. The crude material was purified by column chromatography on silica eluting with 15-20% ethyl acetate/hexane to yield the two title compounds, listed in elution order:

(D13) (131 mg, 50%) was afforded as a colourless oil, δ_{H} (CDCl₃) 1.49 (9H, s), 3.00-3.50 (6H, m), 3.90-4.00 (2H, m), 7.40 (1H, d, J=8.6 Hz), 7.49-7.53 (2H, m), 7.55-7.57 (1H, m), 7.92 (1H, d, J=8.6 Hz), 8.00-8.03 (2H, m), 8.40 (1H, s).

(D14) (49 mg, 19%) was afforded as a white solid, δ_{H} (CDCl₃) 1.49 (9H, s), 3.04-3.06 (4H, m), 3.64-3.66 (4H, m), 6.97 (1H, d, J=8.24), 7.34 (1H, d, J=8.20 Hz), 7.48-7.52 (2H, m), 7.56-7.58 (1H, m), 7.87-7.89 (2H, m), 8.73 (1H, s).

Description 15: 7-(4-tert-Butoxycarbonyl-piperazin-1-yl)-1H-indole-1-carboxylic acid tert-butyl ester (1)15)

To a stirred solution of 4-(1H-indol-7-yl)-piperazine-1-carboxylic acid tert-butyl ester (D3) (1 g, 3.3 mmol) in dry dichloromethane (30 ml) was added di-tert-butyl dicarbonate (1.08 g, 5 mmol) and N,N-dimethylaminopyridine (203 mg, 1.66 mmol) and the reaction mixture stirred under argon for 18 hours. The reaction mixture was concentrated in vacuo and crude material was purified by column chromatography on silica eluting with 20% ethyl acetate/petroleum ether (40-60° C.) to afford the title compound (D15) as a colourless oil. (1.35 g, 100%), δ_{H} (CDCl₃) 1.48 (9H, s), 1.63 (9H, s), 2.98 (4H, m), 3.63 (4H, m), 6.53 (1H, d, J=3.7 Hz), 6.90 (1H, d, J=7.3 Hz), 7.15-7.25 (2H, m), 7.46 (1H, d, J=3.7 Hz). Mass Spectrum: m/z [MH]⁺ 402 (C₂₂H₃₁N₃O₄).

Description 16: 7-(4-tert-Butoxycarbonyl-piperazin-1-yl)-2-methyl-1H-indole-1-carboxylic acid tert-butyl ester (D16)

To a stirred solution of 7-(4-tert-butoxycarbonyl-piperazin-1-yl)-1H-indole-1-carboxylic acid tert-butyl ester (D15) (0.622 g, 1.55 mmol) in tetrahydrofuran (5 ml) at −78° C. under argon was added t-butyllithium dropwise (1.09 ml, 1.9 mmol, 1.7 M solution in hexanes). Stirring was continued for 1 h at the same temperature before the reaction was quenched by addition of iodomethane (0.116 ml, 1.9 mmol). The reaction was then slowly warmed to room temperature and stirred for 18 h. The reaction was poured into aqueous ammonium chloride (10 ml) and the whole was extracted with dichloromethane (20 ml). The organic phase was washed with saturated brine, dried (MgSO₄) and concentrated in vacuo to afford the title compound (D16) (0.653 g, 100%). δ_{H} (CDCl₃) 1.49 (9H, s), 1.64 (9H, s), 2.46 (3H, s), 2.95-2.97 (4H, m), 3.58 (4H, m), 6.24 (1H, s), 6.94 (1H, d, J=7.5 Hz), 7.11 (1H, t, J=7.6 Hz), 7.20 (1H, d, J=7.6 Hz). Mass Spectrum: m/z [MH]⁺ 416 (C₂₃H₃₃N₃O₄).

Description 17: 4-(2-Methyl-1H-indol-7-yl)-piperazine-1-carboxylic acid tert-butyl ester (D17)

To a stirred solution of 7-(4-tert-butoxycarbonyl-piperazin-1-yl)-2-methyl-1H-indole-1-carboxylic acid tert-butyl ester (D16) (473 mg, 1.1 mmol) in 1,4 dioxane (5 ml) was added 4M aqueous hydrochloric acid (5 ml) and the reaction was stirred under argon for 1.5 hours at 80° C. The reaction mixture was concentrated in vacuo to yield a purple solid (285 mg). The solid was dissolved in dichloromethane (10 ml) and to the stirred solution was sequentially added dropwise triethylamine (0.166 ml, 1.2 mmol) and a solution of di-t-butyl dicarbonate (260 mg, 1.2 mmol) in dry dichloromethane (5 ml). The reaction mixture was stirred at ambient temperature under argon for 15 minutes before being diluted with dichloromethane (10 ml) and water (10 ml). After vigorously shaking the mixture, the layer were separated and the organic layer dried (MgSO₄) and concentrated in vacuo. The crude material was purified by column chromatography on silica eluting with 15% ethyl acetate/petroleum ether (40-60° C.) to afford the title compound (D17) (196 mg, 57%), δ_{H} (CDCl₃) 1.50 (9H, s), 2.46 (3H, s), 3.02-3.06 (4H, m), 3.63-3.67 (4H, m), 6.21 (1H, s), 6.75 (1H, d, J=7.6 Hz), 7.00 (1H, t, J=7.7 Hz), 7.26 (1H, d, J=7.2 Hz), 8.00 (1H, br s). Mass Spectrum: m/z [MH]⁺ 316 (C₁₈H₂₅N₃O₂).

Description 18: 4-(2-Methyl-3-phenylsulfanyl-1H-indol-7-yl)-piperazine-1-carboxylic acid tert-butyl ester (D18)

To a stirred suspension of sodium hydride (38 mg, 0.94 mmol) in dry N,N-dimethylformamide (5 ml) was added a solution of 4-(2-methyl-1H-indol-7-yl)-piperazine-1-carboxylic acid tert-butyl ester (D17) (198 mg, 0.63 mmol) in dry N,N-dimethylformamide (5 ml) dropwise over 5 minutes. After stirring at room temperature for 10 minutes under argon, a solution of phenyl disulfide (151 mg, 0.7 mmol) in dry N,N-dimethylformamide (2 ml) was added dropwise and the reaction mixture was stirred at room temperature for a further 4 hours. The reaction mixture was diluted with ether (20 ml) and water (20 ml) and the whole was shaken and separated. The organic layer was dried (MgSO₄) and concentrated in vacuo. The crude material was purified by column chromatography on silica eluting with 20% ethyl acetate/petroleum ether (40-60° C.) to afford the title compound (D18) as an oil. (119 mg, 45%) δ_{H} (CDCl₃) 1.50 (9H, s), 2.54 (3H, s), 3.05-3.09 (4H, m), 3.65-3.69 (4H, m), 6.85 (1H, d, J=7.6 Hz), 7.00-7.06 (4H, m), 7.10-7.17 (2H, m), 7.30 (1H, d, J=7.8 Hz), 8.38 (1H, br s). Mass Spectrum: m/z [MH]⁺ 424 (C₂₄H₂₉N₃O₂S).

Description 19: 4-(2-Methyl-3-phenylsulfonyl-1H-indol-7-yl)piperazine-1-carboxylic acid tert-butyl ester (D19)

To a solution of 4-(2-methyl-3-phenylsulfanyl-1H-indol-7-yl)-piperazine-1-carboxylic acid tert-butyl ester (D18) (119 mg, 0.28 mmol) in dry dichloromethane (5 ml) was added 3-chloroperoxybenzoic acid (291 mg, 0.84 mmol) in a single portion. The reaction mixture was stirred under argon at room temperature for 2 hours before being diluted with dichloromethane (10 ml), water (10 ml) and sodium disulfite (20 ml of 10% w/v solution). The whole was shaken and the organic phase dried (MgSO₄) and concentrated in vacuo. The crude material was purified by column chromatography on silica eluting with 40-50% ethyl acetate/petroleum ether (40-60° C.) gradient to afford the title compound (E)19) (94 mg, 74%). δ_{H} (CDCl₃) 1.48 (9H, s), 2.76 (3H, s), 2.98-3.02 (4H, m), 3.61-3.65 (4H, m), 6.88 (1H, d, J=7.7 Hz), 7.17 (1H, t, J=7.9 Hz), 7.40-7.49 (3H, m), 7.79 (1H, d, J=8.0 Hz), 7.95-7.98 (2H, m), 9.18 (1H, br s). Mass Spectrum: m/z [M−H]⁻ 454 (C₂₄H₂₉N₃O₄S).

Description 20: 4-(1,2-Dimethyl-3-phenylsulfonyl-1H-indole-7-yl)piperazine-1-carboxylic acid tert-butyl ester (D20)

To a solution of 4-(2-methyl-3-phenylsulfonyl-1H-indol-7-yl)-piperazine-1-carboxylic acid tert-butyl ester (D19) (92 mg, 0.2 mmol) in ethanol (2 ml) under argon was added powdered potassium hydroxide (17 mg, 0.3 mmol). After 5 minutes of stirring at room temperature the reaction mixture was concentrated in vacuo. The residual oil was re-suspended in dry acetone (2 ml) and dimethylsulphate (29 μl, 0.3 mmol) was added dropwise. The reaction mixture was stirred at room temperature under argon for 18 h and then diluted with dichloromethane (2×10 ml), washed with water, dried (MgSO₄) and concentrated in vacuo. The crude material was purified by column chromatography on silica eluting with 20-30% ethyl acetate/petroleum ether (40-60° C.) gradient to afford the title compound (D20) (72 mg, 77%), 6H (CDCl₃) 1.49 (9H, s), 2.74 (3H, s), 2.82-2.86 (2H, m), 3.02-3.05 (4H, m), 4.11-4.13 (2H, m), 4.15 (3H, s), 6.98 (1H, d, J=7.4 Hz), 7.15 (1H, t, J=7.9 Hz), 7.41-7.48 (3H, m), 7.92-7.97 (3H, m). Mass Spectrum: m/z [MH]⁺ 470 (C₂₅H₃₁N₃O₄S).

Description 21: 5-Chloro-7-(4-methylpiperazin-1-yl)-1H-indole (D21)

1-(5-Chloro-2-nitro)phenyl-4-methylpiperazine (1)₆) (644 mg, 2.5 mmol), was dissolved in THF (20 ml) and cooled to −45° C. A solution of vinylmagnesium bromide (1M in THF, 8.25 ml) was introduced in one portion at such a rate as to maintain the temperature between −40° C. and −45° C. The mixture was maintained within this temperature range for 30 minutes then poured into saturated aqueous ammonium chloride solution (100 ml). The mixture was extracted with dichloromethane (3×50 ml) and the combined extracts dried (MgSO₄), filtered and evaporated to a brown oil. The residue was co-evaporated with toluene (20 ml) and the resulting oil subjected to purification by chromatography on silica gel (eluting with aqueous ammonia—methanol—dichloromethane) to obtain the title compound (D21) as a brown gum (100 mg, 13%). δ_{H} (CDCl₃) 2.39 (31 s), 2.64 (4H, t, J=4.6 Hz), 3.13 (4H, t, J=4.6 Hz), 6.48 (1H, t, J=2.7 Hz), 6.78 (1H, d, J=1.5 Hz), 7.18 (1H, t, J=2.7 Hz), 7.33 (1H, d, J=1.5 Hz), 8.3 (1H, br s).

Description 22: 5-Chloro-3-(3-chlorophenyl)sulfanyl-7-(4-methyl-piperazin-1-yl)-1H-indole (D22)

A suspension of sodium hydride (40% oil dispersion, 20 mg, 0.5 mmol) in DMF (1 ml) was treated with solution of 5-chloro-7-(4-methylpiperazin-1-yl)-1H-indole (D21) (87 mg, 0.24 mmol) in DMF (1 ml). After effervescence ceased, bis-(3-chlorophenyl)-disulphide (110 mg, 0.39 mmol) was introduced and the mixture stirred for 17 hours. The solution was evaporated and the residue purified by flash chromatography on silica gel (eluting with methanol—dichloromethane—aqueous ammonia) to form the title compound (D22) as a colourless solid (95 mg, 68%). δ_{H} (CDCl₃) 2.40 (3H, s), 2.66 (4H, t, J=4.7 Hz), 3.15 (4H, t, J=4.7 Hz), 6.85 (1H, d, J=1.7 Hz), 6.93 (1H, dd), 7.01-7.10 (3H, m), 7.27 (1H, d, J=1.6 Hz), 7.46 (1H, d, J=2.6 Hz), 8.6 (1H, br s).

### EXAMPLE 1

### 3-(1H-Indole-1-sulfonyl)-7-(4-methyl-piperazin-1-yl)-1H-indole (E1)

3-(1H-Indole-1-sulfonyl)-1H-7-indol-7-ylamine (D2) (0.5 g, 1.6 mmol) was dissolved in n-butanol (20 ml) followed by addition of sodium carbonate (0.85 g, 8.0 mmol) and methchlorethamine hydrochloride (0.47 g, 2.4 mmol). The resulting suspension was heated to reflux for 48 hours. After allowing to cool, the solvent was removed in vacuo, the residue taken up in dichloromethane (100 ml) and washed with saturated sodium hydrogencarbonate solution (2×50 ml). The organic phase was dried (MgSO₄), filtered and the solvent evaporated. The crude was purified by column chromatography on silica, eluting with a methanol/dichloromethane gradient to afford the title compound (E1) (0.25 g, 40%), MS: m/z (M−H)-393, C₂₁H₂₂N₄O₂S requires 394.

### EXAMPLE 2

### 3-(1H-Indole-1-sulfonyl)-7-piperazin-1-yl-1H-indole (E2)

To a solution of 3-(1H-Indole-1-sulfonyl)-7-(4-methyl-piperazin-1-yl)-1H-indole (E1) (0.1 g, 0.25 mmol) in dry 1,2-dichloroethane (5 ml) was added N,N-diisopropylethylamine (0.11 ml, 0.76 mmol) and 1-chloroethyl chloroformate (83 μl, 0.76 mmol). The solution was stirred at 80° C. for 30 minutes, cooled to ambient temperature and then concentrated in vacuo. The residue was dissolved in methanol (10 ml) and the solution heated to reflux for 1 hour. After concentrating the mixture in vacuo, the residue was dissolved in dichloromethane (20 ml) and washed with saturated sodium hydrogencarbonate solution (20 ml) and water (2×10 ml). The organic phase was dried (MgSO₄) and concentrated in vacuo to give a residue which was purified by column chromatography on silica gel, eluting with a methanol/dichloromethane gradient to afford the title compound (E2) (55 mg, 57%), δ_{H} (CD₃OD) 3.22 (4H, br s), 3.42 (4H, br s), 6.62 (1H, d, J=3.7 Hz), 6.92 (1H, d, J=7.7 Hz), 7.12-7.17 (2H, m), 7.25 (1H, t, J=8.2 Hz), 7.49 (1H, d, J=7.8 Hz), 7.59 (1H, d, J=8.1 Hz), 7.76 (1H, d, J=3.7 Hz), 7.96 (1H, d, J=8.3 Hz), 8.09 (1H, s). MS: m/z (M−H) 379, C₂₀H₂₀N₄O₂S requires 380.

### EXAMPLE 3

### 3-(3-Chlorophenyl)sulfonyl-1-methyl-7-piperazin-1-yl-1H-indole hydrochloride (E3)

To a solution of 4-[3-(3-chlorophenyl)sulfonyl-1-methyl-1H-indol-7-yl]piperazine-1-carboxylic acid tert-butyl ester (D5) (52 mg, 0.11 mmol) in 1,4-dioxane (6 ml) was added 3M HCl (6 ml) and the resulting solution heated to 60° C. for 60 minutes. After allowing to cool to ambient temperature, the mixture was concentrated in vacuo to afford the title compound (E3) (39 mg, 90%), δ_{H} (CD₃OD) 3.19 (2H, m), 3.33-3.35 (2H, m), 3.36-3.50 (4H, m), 4.24 (3H, s), 7.20-7.27 (2H, m), 7.52 (1H, t, J=7.9 Hz), 7.59 (1H, m), 7.71 (1H, d, 3=7.8 Hz), 7.92-7.97 (2H, m), 8.00 (1H, s). Mass Spectrum: m/z [MH]⁺ 390 (C₁₉H₂₀ClN₃O₂S).

### EXAMPLES 4-17 (E4E17)

3-(Phenyl)sulfonyl-1-methyl-7-piperazin-1-yl-1H-indole (E4);

3-(2-Fluorophenyl)sulfonyl-1-methyl-7-piperazin-1-yl-1H-indole (ES);

3-(2 Chlorophenyl)sulfonyl-1-methyl-7-piperazin-1-yl-1H-indole (E6);

3-(2-Cyanophenyl)sulfonyl-1-methyl-7-piperazin-1-yl-1H-indole (E7);

3-(3-Fluorophenyl)sulfonyl-1-methyl-7-piperazin-1-yl-1H-indole (E8);

3-(2-Trifluoromethylphenyl)sulfonyl-1-methyl-7-piperazin-1-yl-1H-indole (E9);

3-(Phenyl)sulfonyl-7-piperazin-1-yl-1H-indole (E10);

3-(3-Chlorophenyl)sulfonyl-7-piperazin-1-yl-1H-indole (E11);

3-(2-Fluorophenyl)sulfonyl-7-piperazin-1-yl-1H-indole (E12);

3-(4-Fluorophenyl)sulfonyl-7-piperazin-1-yl-1H-indole (E13);

3-(3-Fluorophenyl)sulfonyl-7-piperazin-1-yl-1H-indole (E14);

3-(2-Pyridyl)sulfonyl-7-piperazin-1-yl-1H-indole (E15);

3-(2-Chlorophenyl)sulfonyl-7-piperazin-1-yl-1H-indole (E16);

3-(2-Cyanophenyl)sulfonyl-7-piperazin-1-yl-1H-indole (E17);

Examples 4-9 were prepared using analogous procedures to those described in D4 followed by D5 and then deprotection in an analogous manner to that described in Example 3 (E3).

Examples 10-17 were prepared using an analogous procedure to that described in D4 followed by deprotection in an analogous manner to that described in Example 3.

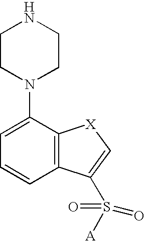

| Example | X | A | m/z [MH]⁺ | Formula |
|---|---|---|---|---|
| E4 | NMe | phenyl | 356 | C₁₉H₂₁N₃SO₂ |
| E5 | NMe | 2-fluorophenyl | 374 | C₁₉H₂₀FN₃SO₂ |
| E6 | NMe | 2-chlorophenyl | 390/392 | C₁₉H₂₀ClN₃SO₂ |
| E7 | NMe | 2-cyanophenyl | 381 | C₂₀H₂₀H₄SO₂ |
| E8 | NMe | 3-fluorophenyl | 374 | C₁₉H₂₀FN₃SO₂ |
| E9 | NMe | 2-tri- | 424 | C₂₀H₂₀F₃N₃SO₂ |
| | | fluoromethylphenyl | | |
| E10 | NH | phenyl | 340(M − H) | C₁₈H₁₉N₃SO₂ |
| E11 | NH | 3-chlorophenyl | 376/378 | C₁₈H₁₈ClN₃SO₂ |
| E12 | NH | 2-fluorophenyl | 360 | C₁₈H₁₈FN₃SO₂ |
| E13 | NH | 4-fluorophenyl | 360 | C₁₈H₁₈FN₃SO₂ |
| E14 | NH | 3-fluorophenyl | 360 | C₁₈H₁₈FN₃SO₂ |
| E15 | NH | 2-pyridyl | 343 | C₁₇H₁₈N₄SO₂ |
| E16 | NH | 2-chlorophenyl | 376/378 | C₁₈H₁₈ClN₃SO₂ |
| E17 | NH | 2-cyanophenyl | 367 | C₁₉H₁₈N₄SO₂ |

### EXAMPLE 18

### 1,2-Dimethyl-3-phenylsulfonyl-7-piperazin-1-yl-1H-indole, hydrochloride (E18)

4-(1,2-Dimethyl-3-phenylsulfonyl-1H-indole-7-yl)piperazine-1-carboxylic acid tert-butyl ester (D20) (64 mg, 0.14 mmol) was dissolved in 1,4-dioxane (1 ml). 4M aqueous hydrochloric acid (1 ml) was added and the reaction mixture stirred at 80° C. under argon for 1.5 h. The solvent was evaporated in vacuo to afford the title compound (E18) as a white solid (45 mg, 79%), 5H ([CD₃]₂SO) 2.72 (3H, s), 3.02-3.39 (8H, m), 4.09 (3H, s), 7.01 (1H, d, J=7.3 Hz), 7.16 (1H, t, J=7.9 Hz), 7.51-7.63 (3H, m), 7.75 (1H, d, J=7.9 Hz), 7.89-7.97 (2H, m), 9.23 (1H, br s), 9.43 (1H, br s). Mass Spectrum: m/z [MH]⁺ 370 (C₂₀H₂₃N₃O₂S).

### EXAMPLE 19

### 4-Methyl-1-(3-phenylsulfonyl-benzo[b]furan-7-yl)piperazine trifluoroacetate (E19)

A mixture of 3-phenylsulfonyl-benzo[b]furan-7-ylamine (D9)(0.273, 1.0 mmol), methchlorethamine hydrochloride (0.243 g, 1.3 mmol) and anhydrous sodium carbonate (0.53 g, 5.0 mmol) in n-butanol (7 ml) were stirred at reflux under argon for 36 h. The mixture was diluted with dichloromethane (25 ml) and washed with water (25 ml). The organic phase was dried (MgSO₄), concentrated to an oil and purified by HPLC chromatography on a 25 cm×21 mm column packed with 12 micron SUPELCOSIL ABZ+eluting with acetonitrile/water/0.1% trifluoroacetic acid solvent gradient to afford the title compound (139) as a solid (60 mg, 0.17 mmol, 17%).

δ_{H} (CD₃OD) 2.98 (3H, s), 3.18 (2H, br t, J=12 Hz), 3.36 (2H, br t, J=12 Hz), 3.63 (2H, br d, J=12 Hz), 3.95 (2H, br d, J=12 Hz), 6.97 (1H, d, J=7.8 Hz), 7.30 (1H, t, J=7.9 Hz), 7.45 (1H, d, 3=8.5 Hz), 7.57-7.67 (3H, m), 8.07 (2H, d, J=7.2 Hz), 8.54 (1H, s).

### EXAMPLE 20

### 1-(3-Phenylsulfonyl-benzo[b]furan-7-yl)piperazine, hydrochloride (E20)

To a solution of 4-methyl-1-(3-phenylsulfonyl-benzo[b]furan-7-yl)piperazine trifluoroacetate (E19) (0.132 g, 0.37 mmol) in 1,2-dichloroethane (1.5 ml) was slowly added 1-chloroethyl chloroformate (0.06 ml, 0.6 mmol) followed by N,N-di-isopropylethylamine (0.1 ml, 0.6 mmol) under argon at ambient temperature. The solution was heated at reflux for 24 h and then further quantities of 1-chloroethyl chloroformate (0.16 ml, 1.5 mmol) and N,N-di-isopropylethylamine (0.26 ml, 1.5 mmol) were added and reflux was maintained for a further 24 h. The reaction mixture was concentrated in vacuo to an oil, which was purified by chromatography over silica gel eluting with a solvent gradient of ethyl acetate/hexane. Column fractions containing the major component (Thin layer chromatography on silica gel plates: Rf 0.58; ethyl acetate/hexane 4:1) were pooled and concentrated in vacuo to an oil. A solution of this oil in methanol (1.5 ml) was heated at reflux for 1 h under argon and then concentrated in vacuo to afford a white solid. The solid was recrystallised from dichloromethane and 1M hydrogen chloride in diethyl ether to give the title compound (E20) (41 mg, 0.11 mmol, 29%).

δ_{H} (CD₃OD) 3.42-3.44 (41, m), 3.52-3.54 (4H, m), 7.00 (1H, d, J=7.8 Hz), 7.30 (1H, t, J=2.7 Hz), 7.44 (1H, d, J=7.4 Hz), 7.58-7.68 (3H, m), 8.08 (2H, 7.3 Hz), 8.57 (1H, s). Mass Spectrum: m/z [MH]⁺343 (C₁₈H₁₈N₂O₃S).

### EXAMPLE 21

### 3-Phenylsulfonyl-7-(1-piperazinyl)benzo[b]thiophene hydrochloride (E21)

7-(4-tert-Butyloxycarbonyl-1-piperazinyl)-3-phenylsulfonyl-benzo[b]thiophene (D12) (45 mg, 0.1 mmol) was dissolved in dioxane (1 ml), and treated with 4M hydrochloric acid (1 ml) with stirring at 80° C. for 5 mins. The solvents were removed and purification was effected by ion exchange chromatography (Varian Mega Bond Elut SCX) using methanol then 15% ammonium hydroxide in methanol. After removal of solvents the product was treated with methanol/ethereal hydrogen chloride and the solvents removed to afford the title compound (E21) (19 mg, 49% yield). δ_{H} (CD₃OD) 8.67 (1H, s) 8.03 (2H, dd, J=8.6 Hz, 1.5 Hz), 7.93 (1H, d, J=8.1 Hz) 7.64-7.47 (4H, m), 7.21 (1H, d, J=7.2 Hz), 3.42 (4H, m) 3.36 (4H, m). Mass Spectrum: m/z [MH]⁺ 359 (C₁₈H₁₈N₂O₂S₂).

### EXAMPLE 22

### 6-Chloro-3-phenylsulfonyl-7-(1-piperazinyl)-benzo[b]thiophene hydrochloride (E22)

7-(4-tert-Butyloxycarbonyl-1-piperazinyl)-6-chloro-3-phenylsulfonyl-benzo[b]thiophene (D13) (131 mg, 0.27 mmol) was dissolved in 1,4-dioxane (2 ml) and aqueous 4M HCl (2 ml) was added. The reaction mixture was stirred under argon at 80° C. for 1.5 h and solvent evaporated in vacuo to afford the title compound (E22) as a white solid (107 mg, 93%), 8H (CD₃OD) 3.32-3.51 (6H, m), 3.84 (2H, m), 7.53-7.67 (4H, m), 8.04-8.07 (3H, m), 8.71 (1H, s). Mass Spectrum: m/z [MH]⁺ 393, 395 (C₁₈H₁₇ClN₂O₂S₂).

### EXAMPLE 23

### 4-Chloro-3-phenylsulfonyl-7-(1-piperazinyl)-benzo[b]thiophene hydrochloride (E23)

The title compound (E23) was prepared in quantitative yield (37 mg) from 74-tert-butyloxycarbonyl-1-piperazinyl)₄-chloro 3-phenylsulfonyl-benzo[b]thiophene (D14) (42 mg, 0.086 mmol) as described in Example 1. SH ([CD₃]₂SO) 3.29-3.34 (8H, m), 7.25 (1H, d, J=8.2 Hz), 7.48 (1H, d, J=8.2 Hz), 7.60-7.66 (2H, m), 7.69-7.73 (1H, m), 7.83-7.85 (2H, m), 9.06 (3H, s). Mass Spectrum: m/z [MH]⁺ 393, 395 (C₁₈H₁₇ClN₂O₂S₂).

### EXAMPLE 24

### 4,6-Dichloro-3-phenylsulfonyl-7-(1-piperazinyl)-benzo[b]thiophene hydrochloride (E24)

To a solution of 7-(4-tert-butyloxycarbonyl-1-piperazinyl)-3-phenylsulfonyl-benzo[b]thiophene (D12) (285 mg, 0.62 mmol) in glacial acetic acid (10 ml) was added N-chlorosuccininmide (166 mg, 1.24 mmol) portionwise over 10 minutes. Reaction mixture was heated at 600 C for 18 h under argon. The reaction mixture was then cooled to room temperature, diluted with dichloromethane (20 ml), water (10 ml) and adjusted to pH 8 with saturated sodium hydrogen carbonate solution. The organic layer was separated, washed with brine, dried (MgSO₄) and concentrated int vacuo to afford an oil, which was purified by column chromatography on silica gel eluting with a solvent gradient of dichloromethane/methanol followed by HPLC chromatography on a 25 cm×21 mm column packed with 12 micron SUPELCOSIL ABZ+, eluting with an acetonitrile/water/0.1% trifluoroacetic acid solvent gradient. The resulting solid was dissolved in MeOH (5 ml) and treated with 1M hydrogen chloride in diethyl ether (0.2 ml, 4 equiv.) to afford the title compound (E24) as a white solid (22 mg, 8%) δ_{H} (CD₃OD) 3.35-3.62 (6H, m), 3.91-4.00 (2H, m), 7.62 (1H, s), 7.64-7.80 (3H, m), 7.95-7.99 (2H, m), 8.96 (1H, s). Mass Spectrum: m/z [MH]⁺ 427 429, 431 (C₁₈H₁₆Cl₂N₂O₂%).

### EXAMPLE 25

### 5-Chloro-3-(3-chlorophenyl)sulfonyl-7-(4-methyl-piperazin-1-yl)-1H-indole (E25)

A solution of 5-chloro-3-(3-chlorophenyl)sulfanyl-7-(4-methyl-piperazin-1-yl)-1H-indole (D22) (90 mg, 0.21 mmol) in trifluoroacetic acid (1.5 ml) was treated with hydrogen peroxide (27% aqueous, 100 mg), and the mixture stirred at ambient for 2 hours. The mixture was poured into saturated sodium sulphite (5 ml), treated with saturated aqueous sodium carbonate (5 ml) and extracted into dichloromethane (3×10 ml). The combined organic extracts were dried (MgSO₄), filtered and evaporated and the residue purified by flash chromatography on silica gel (eluting with dichloromethane—methanol—aqueous ammonia) to give the title compound (E25) (39 mg, 40%) as a colourless solid. δ_{H} (CDCl₃) 2.38 (3H, s), 2.64 (4H, t, 4.8 Hz), 3.09 (4H, t, 4.8 Hz), 6.9 (1H, d, J=1.7 Hz), 7.43 (1H, t, J=7.8 Hz), 7.50 (1H, dd, J=1.2, 3 Hz), 7.60 (1H, d, J=1.7 Hz), 7.88-7.95 (3H, m), 8.9 (1H, br s); m/z [MH]⁺ 424, 426,428, (C₁₉H₁₉Cl₂N₃O₂S).

### EXAMPLE 26

### 5-Chloro-3-(3-chlorophenyl)sulfonyl-7-piperazin-1-yl-1H-indole (E26)

A solution of 5-chloro-3-(3-chlorophenyl)sulfonyl-7-(4-methyl-piperazin-1-yl)-1H-indole (E25) (40 mg, 0.09 mmol) in dichloromethane (0.5 ml) was treated with l-chloroethyl chloroformate (50 μl) and Hunig's base (100 μl). After 2 hours, methanol (5 ml) and potassium carbonate (70 mg, 0.5 mmol) were added and the mixture heated to reflux for 2 hours. The cooled mixture was evaporated, treated with acetic acid (0.5 ml), evaporated, and the residue subjected to flash chromatography on silica gel (eluting with dichloromethane—methanol aqueous ammonia) to afford the title compound as a colourless solid (E26) (30 mg, 78%) δ_{H} (CDCl₃+trace Hunig's base) 3.04 (4H, br s), 3.0 (2H, br s), 3.18 (4H, br s), 6.79 (1H, d, J=1.6 Hz), 7.33-7.42 (2H, m), 7.53 (1H, d, J=1.6 Hz), 7.81-7.87 (3H, m); Mass Spectrum: m/z [MH]⁺ 410, 412, 414, (C₁₈H₁₇Cl₂N₃O₂S).

Pharmacological Data

Compounds can be tested following the procedures outlined in WO98/27081.

The compounds of Examples E1-E26 were tested and all showed good affinity for the 5-HT₆ receptor, having pKi values >8.0 at human cloned 5-HT₆ receptors.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

1. A compound of formula (I) or a pharmaceutically acceptable salt thereof: 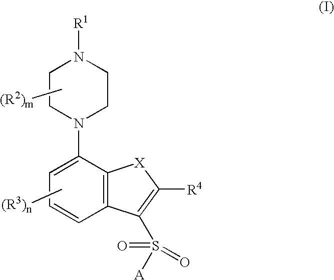
wherein:
R¹ and R² independently represent hydrogen or C₁₋₆ alkyl or R¹ is linked to R² to form a group (CH₂)₂, (CH₂)₃ or (CH₂)₄;
R³ independently represents hydrogen, halogen, cyano, —CF₃, —CF₃O, C₁₋₆ alkyl, C₁₋₆ alkoxy, C₁₋₆ alkanoyl or a group —CONR⁵R⁶;
R⁴ represents hydrogen or C₁₋₆ alkyl;
R⁵ and R⁶ independently represent hydrogen or C₁₋₆ alkyl or together may be fused to form a 5- to 7-membered aromatic or non-aromatic heterocyclic ring optionally interrupted by an O or S atom;
m represents an integer from 1 to 4, such that wherein m is an integer greater than 1, said R² groups may optionally be linked to form a group CH₂, (CH₂)₂ or (CH₂)₃;
n represents an integer from 1 to 3;
X represents NH, N—C₁₋₆ alkyl, O or S;
A represents a group —Ar¹ or —Ar²Ar³;
Ar¹, Ar² and Ar³ independently represent an aryl group or a heteroaryl group, both of which may be optionally substituted by one or more substituents which may be the same or different, and which are selected from the group consisting of halogen, hydroxy, cyano, nitro, trifluoromethyl, trifluoromethoxy, C₁₋₆ alkyl, trifluoromethanesulfonyloxy, pentafluoroethyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, arylsulfonyl, arylsulfonyloxy, arylsulfonylC₁₋₆ alkyl, C₁₋₆ alkylsulfonamido, C₁₋₆ alkylamido, C₁₋₆ alkylsulfonamidoC₁₋₆ alkyl, C₁₋₆ alkylamidoC₁₋₆ alkyl, arylsulfonamido, arylcarboxamido, arylsulfonamidoC₁₋₆ alkyl, arylcarboxamidoC₁₋₆ alkyl, aroyl, aroylC₁₋₆ alkyl, arylC₁₋₆ alkanoyl, or a group CONR⁷R⁸ or SO₂NR⁷R⁸, wherein R⁷ and R⁸ independently represent hydrogen or C₁₋₆ alkyl or together may be fused to form a 5- to 7-membered aromatic or non-aromatic heterocyclic ring optionally interrupted by an O or S atom;
or solvates thereof.

2. A compound according to claim 1 which is:
3-(1H-Indole-1-sulfonyl)-7-(4-methyl-piperazin-1-yl)-1H-indole;
3-(1H-Indole-1-sulfonyl)-7-piperazin-1-yl-1H-indole;
3-(3-Chlorophenyl)sulfonyl-1-methyl-7-piperazin-1-yl-1H-indole hydrochloride;
3-(Phenyl)sulfonyl-1-methyl-7-piperazin-1-yl-1H-indole;
3-(2-Fluorophenyl)sulfonyl-1-methyl-7-piperazin-1-yl-1H-indole;
3-(2-Chlorophenyl)sulfonyl-1-methyl-7-piperazin-1-yl-1H-indole;
3-(2-Cyanophenyl)sulfonyl-1-methyl-7-piperazin-1-yl-1H-indole;
3-(3-Fluorophenyl)sulfonyl-1-methyl-7-piperazin-1-yl-1H-indole;
3-(2-Trifluoromethylphenyl)sulfonyl-1-methyl-7-piperazin-1-yl-1H-indole;
3-(Phenyl)sulfonyl-7-piperazin-1-yl-1H-indole;
3-(3-Chlorophenyl)sulfonyl-7-piperazin-1-yl-1H-indole;
3-(2-Fluorophenyl)sulfonyl-7-piperazin-1-yl-1H-indole;
3-(4-Fluorophenyl)sulfonyl-7-piperazin-1-yl-1H-indole;
3-(3-Fluorophenyl)sulfonyl-7-piperazin-1-yl-1H-indole;
3-(2-Pyridyl)sulfonyl-7-piperazin-1-yl-1H-indole;
3-(2-Chlorophenyl)sulfonyl-7-piperazin-1-yl-1H-indole;
3-(2-Cyanophenyl)sulfonyl-7-piperazin-1-yl-1H-indole;
1,2-Dimethyl-3-phenylsulfonyl-7-piperazin-1-yl-1H-indole, hydrochloride;
4-Methyl-1-(3-phenylsulfonyl-benzo[b]furan-7-yl)piperazine trifluoroacetate;
1-(3-Phenylsulfonyl-benzo[b]furan-7-yl)piperazine, hydrochloride;
3-Phenylsulfonyl-7-(1-piperazinyl)benzo[b]thiophene hydrochloride;
6-Chloro-3-phenylsulfonyl-7-(1-piperazinyl)-benzo[b]thiophene hydrochloride;
4-Chloro-3-phenylsulfonyl-7-(1-piperazinyl)-benzo[b]thiophene hydrochloride;
4,6-Dichloro-3-phenylsulfonyl-7-(1-piperazinyl)-benzo[b]thiophene hydrochloride;
5-Chloro-3-(3-chlorophenyl)sulfonyl-7-(4-methyl-piperazin-1-yl)-1H-indole;
5-Chloro-3-(3-chlorophenyl)sulfonyl-7-piperazin-1-yl-1H-indole;
or a pharmaceutically acceptable salt thereof.

3. A process for the preparation of a compound of formula (I) or a pharmaceutically acceptable salt thereof, which process comprises:
(a) reacting a compound of formula (II) 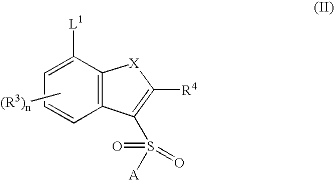
with a compound of formula (III) 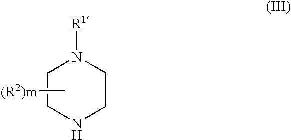
wherein R¹ is as defined in claim 1 for R¹ or an N-protecting group, R², R³, R⁴, A, X, m and n are as defined in claim 1 and L¹ represents a suitable leaving group; or
(b) forming a compound of formula (I) wherein R² represents hydrogen which comprises reacting a compound of formula (IV) 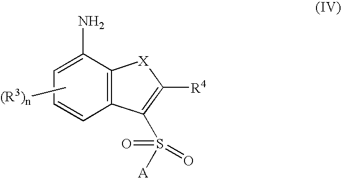
with a compound of formula (V) 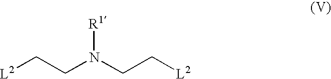
wherein R^{1′} is as defined in claim 1 for R¹ or an N-protecting group, R³, R⁴, X, A and n are as defined in claim 1 and L² represents a suitable leaving group; or
(c) oxidation of a compound of formula (VI) 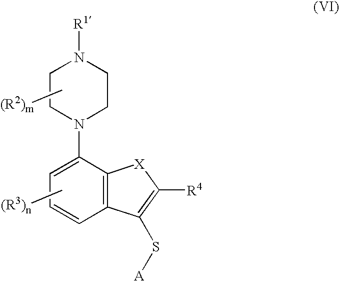
wherein R^{1′} is as defined in claim 1 for R¹ or an N-protecting group and R², R³, R⁴, A, m, n and X are as defined in claim 1; or
(d) deprotecting a compound of formula (I) which is protected;
and optionally thereafter interconversion to other compounds of formula (I).

4. A pharmaceutical composition which comprises a compound according to claim 1 and a pharmaceutically acceptable carrier or excipient.

5. A method of treating depression, anxiety, Alzheimers disease, age related cognitive decline, ADHD, obesity, mild cognitive impairment and schizophrenia which comprises administering a safe and therapeutically effective amount to a patient in need thereof of a compound of formula (I) as defined in claim 1 or a pharmaceutically acceptable salt thereof.

6-15. (canceled).
